(19) United States
(12) Patent Application Publication
(10) Pub. No.: US 2007/0264331 A1
(43) Pub. Date: Nov. 15, 2007

(54) Stable pharmaceutical composition of immediate-release glimepiride and extended-release metformin

(75) Inventors: Antonio Santiago Regalado, Delegacion Iztapalapa (MX); Sixto Serafin Espinoza Leon, Toluca (MX)

Correspondence Address: STERNE, KESSLER, GOLDSTEIN & FOX P.L.L.C., 1100 NEW YORK AVENUE, N.W., WASHINGTON, DC, 20005, US

(73) Assignee: Laboratorios Silanes, S.A de C.V., Delegacion Benito Juarez (MX)

(21) Appl. No.: 11/517,567

(22) Filed: Sep. 8, 2006

(30) Foreign Application Priority Data
Sep. 8, 2005 (MX) PA/A/2005/009633

(51) Int. Cl. A61K 9/22; A61K 31/155; A61K 31/4015; A61P 3/10; A61K 31/64

(52) U.S. Cl. 424/468; 514/423; 514/592; 514/635

(57) ABSTRACT

This invention is directed to a pharmaceutical composition in the form of a tablet with improved stability, as well as the process for obtaining said composition. The tablet of the present invention comprises two active ingredients comprising two oral hypoglycemic agents: one phase with a sulphonylurea, such as immediate release Glimepiride, and a second phase with a biguanide, such as extended-release Metformin hydrochloride (Metformin HCl). The biphasic tablet, which can include over 500 mg of Metformin HCl (i.e. up to 1,000 or 1,500 mg, depending on the daily requirements of each patient), is to be orally administered once or twice a day. The combination of these hypoglycemic agents has a synergic effect and therefore a greater effectiveness in controlling the blood glucose level in patients with diabetes mellitus, type 2.

### TECHNICAL FIELD OF THE INVENTION

This invention is directed to a pharmaceutical composition in the form of a tablet with improved stability, as well as the process for preparing said composition. The pharmaceutical composition of the present invention combines the therapeutic action of two oral hypoglycemic agents: one phase with a sulphonylurea, such as immediate-release Glimepiride, and a second phase with a biguanide, such as extended-release Metformin hydrochloride (Metformin HCl). The biphasic tablet, which can include over 500 mg of Metformin HCl (i.e. up to 1,000 mg or 1,500 mg, depending on the daily requirements of each patient), is to be orally administered once or twice a day. The combination of these hypoglycemic agents has a synergic effect and therefore a higher effectiveness in controlling the blood glucose levels in patients with diabetes mellitus, type 2.

### BACKGROUND OF THE INVENTION

Because of their inherent physicochemical properties, it is particularly difficult to formulate combinations of two biologically-active agents. This application describes a novel invention comprising a single-dose pharmaceutical composition with proven stability, in the form of a tablet. The tablet comprises a very thin layer or coat as a phase containing the first drug to be released immediately. The second phase comprises a lower concentration of a second drug, and is located in the core of the tablet.

Generally, biphasic tablets of this type have a disproportion between the two drugs contained therein. During manufacture, this disproportion makes it difficult to obtain a product with uniform drug contents.

This invention proposes a new stable pharmaceutical composition designed to carry a sulphonylurea, such as Glimepiride, as the first drug for immediate release, and a biguanide, such as Metformin HCl, as the second drug for extended release for 12 hours. This is a uniform composition and, as described in detail, it has proper pharmaceutical stability because there is no possibility of the two drugs mixing during storage. Further, no premature release of the second drug is observed during its release according to desired parameters; not even when using Metformin HCl at dosages of 1,000 to 1,500 mg as the second drug, as exemplified. The present invention is also directed the process for obtaining the described composition. The process of the present invention is advantageous because it requires an amount of labor and skill that is amenable to industrial production, which is translated in advantages in production costs.

This invention refers particularly to a pharmaceutical composition in the form of a tablet comprising as active ingredients a first drug: sulphonylurea Glimepiride, and a second drug: biguanide Metformin HCl.

The use of sulphonylureas as treatment for Diabetes, type 2, is well established as an effective method for controlling hyperglycemia. At the molecular level, the sulphonylureas act on the pancreatic β-cell receptors, known as SUR, activating them and closing an ATP-dependant potassium channel, decreases the influence of potassium and therefore causes a depolarization of the membrane. This depolarization of the membrane causes an increase in the flow of calcium to the interior of the cell activating the cytoskeleton, further causing a translocation of the secement granules, resulting in the release of insulin by exocytosis.

Another treatment which use has been recently disclosed is that of Biguanide Metformin HCl which acts effectively not only in controlling hyperglycemia but also in preventing it. Metformin HCl has a mechanism of action different than sulphonylureas, increasing the sensitivity to insulin in hepatic and peripheral (mainly muscular) tissues. Metformin HCl inhibits the gluconeogenesis and hepatic glycogenolysis. At the cellular level, a greater sensitivity to insulin is explained by the increase of activity induced on the tirosine kynase postreceptor and the consequential increase in the number and activity of GLUT4 transporters.

However, about 75% of patients with Type 2 Diabetes who are treated with sulphonylureas aren't able to adjust their glucose levels to desired levels and require supplementing the treatment with a second oral agent. Also, most of the patients who are treated with a single-drug such as sulphonylurea require, after some years, an additional drug to help with the control therapy and attain glycemic control. The causes of this loss of effectiveness have not yet been determined. One possible explanation is that a gradual deterioration of the pancreas prevents it from maintaining an insulin excretion rate for an extended period of time, which is exacerbated by the constant and long-term stimulation caused by the therapy with sulphonylurea. However, Metformin HCl therapy, whose activity is not based in the stimulation of β-cells, also shows a lack of response after some time of prolonged use, which would contradict the explanation given to the loss of response of sulphonylureas.

On the other hand, it has been discovered that a combination therapy of sulphonylureas and Metformin HCl is more effective than the monotherapy with any of these two drugs. It has been widely proven that the hypoglycemic action of Metformin HCl is completely additive to the one of sulphonylureas (de Fronzo R A, Goodman A M Yn. Engl. J. Med. 333,541 (1995)).

It has been also reported that when the monotherapy with sulphonylureas doesn't attain the desired level it should not be discontinued to be substituted with monotherapy of Metformin HCl because this will diminish the blood glucose level below the levels observed with monotherapy of sulphonylureas (Rosenstock J, Samols E., Muchmore D B, Sheneider J. Diabetes Care, 19, 1194 (1996); Gasber A J, Duncan T G, Goodman A M, Mills D J, Rohtf J L, Amer. J. Med. 103, 491 (1997)).

It is generally known that because Diabetes Mellitus is a chronic disease, the patients with good initial response to oral agents will eventually require a second drug to attain the desired glycemic control. The addition of Metformin HCl to the therapy of sulphonylureas, or vice-versa, as already mentioned, gives an additional response not only in the decrease of glucose but also in the decrease of lipids (Hermann L S Schersten B, Bitzen P O, Kjellstrom T, Lindgarde F, Melander A. Diabetes Care, 17, 1100 (1994)).

Although the proportions of sulphonylurea and Metformin HCl used in the preceding studies vary, reaching an optimum therapeutic effect is truly complicated due to quantitative disproportion of the drugs in the combinations used. One of the most important reasons for this large disproportion is the diversity of the drug requirements in congruence with the diversity of metabolic conditions of diabetic patients, as well as due to different progression of the disease in individual patients that requires keeping the required doses within specific ranges.

To achieve a better contribution to the requirements of the drugs mentioned, this invention includes the development of a product with doses of 2 and 4 mg of immediate-release Glimepiride and doses between 850 and 1,000 mg of extended-release Metformin HCl. With this mechanism of release the duration of the therapeutic effect is increased because it keeps the levels of the active ingredients in the bloodstream constant throughout the release, allowing the reduction of both the frequency of dosages (one tablet, twice a day) and the manifestation of side reactions, providing more convenience to the patient and offering a more secure, efficient and reliable treatment.

Sulphonylurea glibenclamide and Metformin HCl have been commonly used simultaneously, providing better effectiveness than when used alone and offering the possibility of using a lower amount of drug, reducing side effects such as anorexia, nausea, vomit and diarrhea. Among these are the following:

In several documents relating to the field of the present invention, such as patent applications WO 97/17975 dated May 27, 1997; U.S. Ser. No. 09/353,141, and WO 01/32158 dated Nov. 13, 1999, as well as in documents: Vigneri et al., Diabetes y Metabolism 17, 232-234 (1991), Higginbotham et al., Med. J. Austr. 154-156 (1979), the handling of several proportions of Sulphonylurea to Metformin HCl is disclosed; however, reaching the optimum therapeutic effect is complicated because quantitative disproportion of the drugs in the combinations used. One of the most important reasons for such disproportion is the diversity of drug requirements due to the diversity of metabolic conditions of the patients with diabetes and the different evolution of the disease in each person which forces to keep the doses required within specific ranges.

Currently, Laboratorios Silanes S.A. de C.V. has a product line called Glimetal® (Metformin HCl 1,000 mg—Glimepiride 4 mg or 2 mg and Metformin HCl 500 mg—Glimepiride 1 mg) that provides a viable alternative for improved control and prevention of type 2 diabetes. The release profile of Glimetal® is shown in the following graph: See FIG. 1.

Unlike Glimetal®, the dissolution-release profile of the composition described in this invention offers a 100%-availability of Glimepiride in the first 60 minutes and a slow extended release of Metformin HCl for about 12 hours.

Another document in the field of the present invention is patent application WO 2004/045622 A1, to Trehan A. et al, dated Jun. 3, 2004, which discloses a pharmaceutical composition that combines an extended-release biguanide and an immediate-release sulphonylurea in separate doses, but administered at the same time. The composition disclosed by Trehan is a tablet with a 500-mg Metformin HCl core, an insulating layer or cover of 15.6 mg with Hydroxypropyl methyl-cellulose E5 and 4.8 mg of Polyethylene glycol 4,000, among other components, and a coating with Glimepiride equivalent to 2 mg as active. The release percentage of Glimepiride in the composition revealed by Trehan in an in vitro dissolution assay is 92% at 15 minutes, 101% at 30 minutes, and 105% at 45 minutes. The release percentage of Metformin HCl in the composition revealed by Trehan in an in vitro dissolution assay is 28% at hour 1, 64% at hour 4, 91% at hour 8, and 100% at hour 12.

It is evident that the insulating coatings in the tablet revealed by Trehan and the one described in this invention have different formulations. According to the present invention, the ethyl cellulose present in the insulating layer, unlike hydroxypropyl methyl-cellulose, is completely hydrophobic and therefore confers stability to the tablet because it avoids the diffusion of Glimepiride to the core containing Metformin HCl. In the Trehan application, no stability tests are revealed for the tablet and no examples given, or at least suggested, where more than 500 mg of Metformin HCl are used. The invention tablet shows that compositions with over 500 mg (1,000 mg of Metformin HCL as exemplified later) can be formulated, even up to 1,500 mg, still keeping the release profiles and the stability properties wanted.

One comparison point between the release profile of Trehan's composition and the composition of this invention is at 8 hours, when Trehan's composition has released 91%, whereas the composition of the present invention has released 80% at 8 hours, reflecting a more controlled release.

Another document in the field of the present invention is the patent of Lim J. et al U.S. Pat. No. 6,682,759, dated on Jan. 27, 2004, which discloses an oral tablet containing a system of immediate-release of a drug, including Glimepiride, and another drug, including Metformin hydrochloride, with extended release. The immediate release is achieved with drug particles equal or smaller than 10 microns in diameter applied in the layer or coating of a core housing the extended-release drug. This document only exemplifies the process for obtaining tablets containing 500 mg of Metformin HCl and 2.10 mg of Glimepiride. According to the procedure revealed by Lim in this document, Glimepiride is formulated in a coating surrounding the core of Metformin, but there is no intermediate insulating layer between the first and second drugs. However, the tablets revealed in Lim's document don't have an intermediate seal between the coating of the first drug and the second drug in the core; no examples are provided for quantities over 500 mg of Metformin HCl and no evidence of the release or dissolution profiles for the tablets nor evidence of their stability are given.

Another document relating to the field of the present invention is the American patent application of Castan C. et al. published under number US 2004/0219212 A1 and dated Nov. 4, 2004, which discloses a pharmaceutical composition of daily single-dose tablets formed by 50- and 1,000-micron micro-capsules with a core of Metformin HCl (500 mg). The tablet also has sulphonylurea glibenclamide (1.5 mg) in micro-particles that are 200- 500-microns in diameter. The micro-capsules are covered with a cellulose film that allows an extended release of Metformin HCl. This mixture of both drugs is located in a gelatin capsule.

The dissolution profile for the tablets disclosed by Castan shows that Metformin HCl micro-capsules have released 40% at 2 hours, 71% at 4 hours, 92% at 8 hours, and 100% at 16 hours; the micro-particles of glibenclamide are released 100% during the first 8 minutes (as shown in FIG. 3 of Castan's document).

The pharmaceutical composition of the Castan application discloses a release of Metformin HCl extending over 12 hours and the sulphonylurea used is released in the first 8 minutes. The release profile of the Metformin is comparable with the one shown in this invention. However, the pharmaceutical composition based on micro-capsules, like the one revealed by Castan, doesn't allow the use of larger concentrations of either drug because the limit of pharmaceutically acceptable volumes. The pharmaceutical form of the invention herein, as opposed to the one revealed by Castan, doesn't have volume problems when using drug doses between the range 0.2 or less, to 1,000 or 1,500 mg. No capsule stability data is shown.

Another document relating to the field of the present invention is the patent application of Hussain J. et al. published under number US 2003/0187074 disclosing a release system for treating patients with diabetes mellitus, type 2. This system consists of a two-layered pharmaceutical form, one with a biguanide, including Metformin HCl, of controlled release depending on the pH environment, and the other layer of sulphonylurea, with Glimepiride being mentioned.

The tablets revealed by Hussain are biphasic systems with granule-core tablets or bi-layered tablets with a granule layer. Such granules have a Metformin HCl core covered with a layer of ethyl-cellulose. The granules are 850 μm, they are compressed and covered by a layer of the second drug (rosiglitazone) of immediate release. The release profile of Metformin is 45.7% at the first hour, 73.7% at hour 3, 89.00% at hour 6, without showing a release extension at hour 12. The release profile of rosiglitazone shown in Hussain's tablets was 90.95% at 0.5 minutes and 103.74% at 10 minutes. The bi-layered tablets have glizipide as immediate release drug and its release profile shows 68.52% after 2 minutes, 89.15% and it stays at 95.2% from 10 to 30 minutes. Hussain's pharmaceutical compositions don't have an insulating coating between both active ingredients. The coating is not designed as in the invention herein; the release profile is not as extended, and it doesn't show stability data.

### BRIEF DESCRIPTION OF THE INVENTION

One embodiment of the present invention is a solid pharmaceutical composition in the form of a tablet for oral administration comprising a core or matrix containing an extended-release biguanide, an insulating layer or coating made of a hydrophobic polymer like ethyl-cellulose, and a coating containing an immediate-release sulphonylurea whose pharmaceutical stability prevents both drugs from mixing and, during the release, avoids premature release of the extended-release active ingredient or delay in the immediate release active ingredient.

Another embodiment of the present invention is a pharmaceutical composition comprising one or more of the following active drugs: a biguanide, such as Metformin, fenformin, or buformin; and a sulphonylurea such as Glimepiride, glipizide or glyburide, glibornuride, glisoxepide, gliclazide, acetohexamide, clopropamide, tolazamide, or tolbutamide.

Another embodiment of the present invention comprises a pharmaceutical composition wherein the biguanide may be released for a period of between approximately 6 and 24 hours, preferably between about 8 and 12 hours, after oral administration.

Another embodiment of the present invention is a pharmaceutical composition in the form of a tablet for oral administration, preferably in one or two doses daily, depending on the patient's requirements.

Yet another embodiment of the present invention is a pharmaceutical composition comprising a combination of two active ingredients. The core or matrix of the tablet of said composition has, besides the active ingredient, an excipient whose components allow an extended release and which includes a relative hydrosolubility polymer like hydroxypropyl methyl-cellulose K-100 M, besides microcrystalline cellulose PH 101, polyvidone K90 and also colloidal silicon dioxide and magnesium stearate.

Another embodiment of the present invention is a pharmaceutical composition in the form of a tablet for oral administration, wherein the insulating coating has a polymer and forms a thin layer with low hydrosolubility, like ethyl-cellulose E 7-7050 clear, combined with Opadry, preferably Opadry clear YS-1-7006.

Yet another embodiment of the present invention is a pharmaceutical composition in the form of a tablet for oral administration, wherein the external coating has the active ingredient and excipients to allow its immediate release and contains a surfactant agent such as sodium lauryl sulphate, colloidal silicon dioxide, an Opadry and a surfactant agent with solubilizing action from the group consisting of polyethylene glycol, propylene glycol or simethicone. The tablet may also have a shiny coating made of Opadry in purified water.

Yet another embodiment of the present invention is directed to a process comprising: a) obtaining the formulation of the matrix or core of the tablet in granulating stage using conventional methods containing 19 to 40% w/w of Hydroxypropyl methyl-cellulose K-100 M and 50 to 70% w/w of the second drug of extended release, that in this case is a biguanide, with an acceptable humidity range; b) conforming the matrix tablet using conventional methods; c) dispersing the insulating coating, which contains between 0.6 and 1% of ethyl-cellulose E-7-7050, over the core using conventional methods; d) dispersing the coating with the first drug of immediate release, that in this case is a sulphonylurea, in an excipient compatible with the physicochemical properties of the first drug like a hydrophobic polymer, over the insulating coating.

Another embodiment of the present invention is directed to a process comprising: a) obtaining the formulation of the matrix or core of the tablet in granulating stage using conventional methods containing 19 to 40% of Hydroxypropyl methyl-cellulose K-100 M and 50 to 70% w/w of extended-release Metformin hydrochloride with an acceptable humidity range; b) conforming the tablet of the matrix using conventional methods; c) dispersing the insulating coating, which contains between 0.6 to 1% of ethyl-cellulose E-7-7050, over the core using conventional methods; d) dispersing the coating with the first drug of immediate release, namely Glimepiride with 0.17% plus an excess of 10%, in an excipient compatible with the physicochemical properties of the first drug like simethicone, sodium lauryl sulphate, opadry and colloidal silicon dioxide.

### BRIEF DESCRIPTION OF FIGURES

FIG. 1 shows one of the release profiles of a commercial modality of the pharmaceutical composition of this invention.

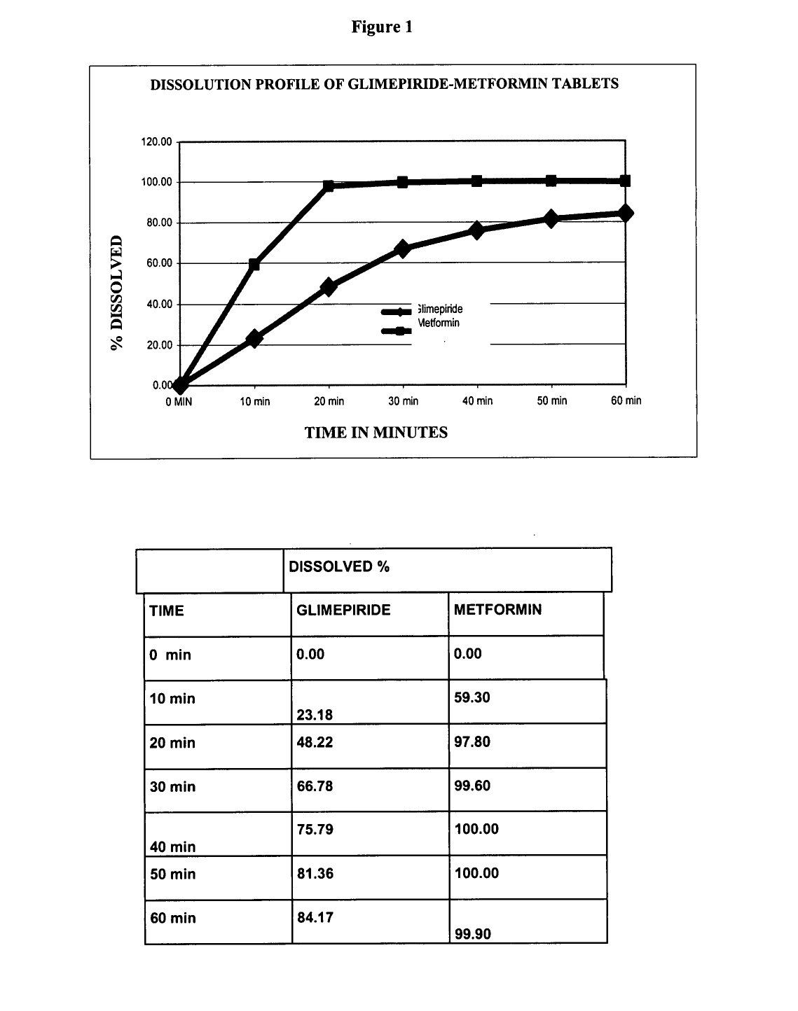

FIG. 2 shows a graph of another release profile of the active ingredients according to this invention.

FIG. 3 shows a graph with the results of dissolution tests performed on the composition of this invention without the insulating coating.

### DETAILED DESCRIPTION OF THIS INVENTION

This description refers to a new invention consisting of single-dose pharmaceutical form of proven stability in the form or a tablet with a very thin layer or coating of a phase containing the first drug to be released immediately and with very low concentration compared to the concentration of the second drug of the second phase located in the core of the tablet.

Generally, biphasic tablets of this type present a disproportion between the two drugs, and during manufacture, this disproportion makes it difficult to obtain a product whose drug contents are uniform.

This invention proposes a new stable pharmaceutical composition designed to contain combinations of two active ingredients in ranges that allow the control of hyperglycemia in patients with diabetes mellitus, type 2, in one or two oral administrations daily. The pharmaceutical composition comprises two biologically active agents in separated phases that have substantial differences in their inherent physicochemical properties and that must have significant disproportions regarding concentration, weight and volume.

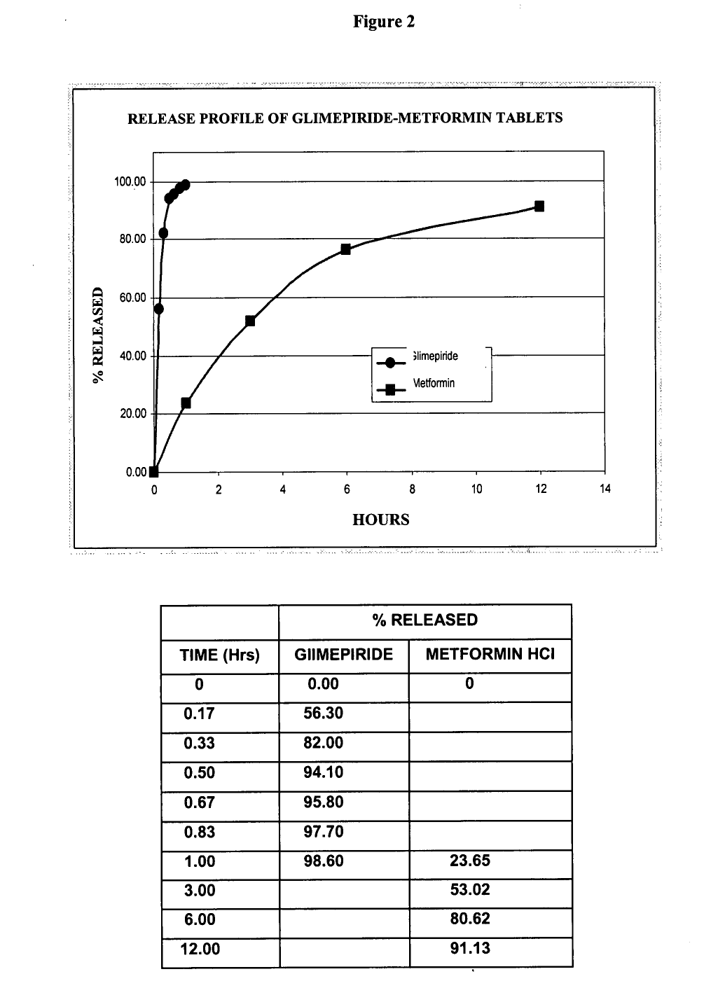

The new pharmaceutical composition contains as active ingredient a sulphonylurea (Glimepiride) that is highly hydrophobic and not soluble in water. This is the first drug of immediate release which is combined with biguanide (Metformin hydrochloride salt or other Metformin salts). Biguanide is highly soluble in water and is included as the second drug of extended release. This combination is a uniform composition as described in detail. Regardless the proportional differences and the differences of physicochemical properties, the composition has proper pharmaceutical stability because there is no possibility of the two drugs mixing during storage. No premature release of Metformin is observed during its release, nor any delay in the release of Glimepiride according to desired parameters, not even when using Metformin HCl concentrations between 1,000 and 1,500 mg. Tablets can be formulated with a weight ratio between Glimepiride and Metformin preferably selected from the following group: 1/500, 2/850, 4/850, 2/1,000, 4/1,000. The release profile of the pharmaceutical composition of the invention is shown in FIG. 2.

The formulation of the invention composition is detailed in the following table.

TABLE I
Qualitative Formula

| | |
|---|---|
| COMPONENTS | mg/Tablet |

Core with Extended-release Active

| | |
|---|---|
| Metformin, hydrochloride | 250.0 to 1500.0 |
| Microcrystalline Cellulose PH 101 | 10.0 to 100 |
| Polyvidone K 90 | 30.0 to 150.0 |
| Hydroxypropyl methyl-cellulose K-100 M | 150.0 to 500.0 |
| Colloidal silicon dioxide | 1.0 to 50.0 |
| Magnesium stearate | 5.0 to 50.0 |

Insulating Coating

| | |
|---|---|
| Ethyl-cellulose E-7-7050 Clear | 5.0 to 50.0 |
| Opadry clear YS-1-7006 | 0.2 to 10.0 |

Coating with immediate release active

| | |
|---|---|
| Glimepiride | 0.2 to 10.0 |
| Sodium lauryl sulphate | 0.2 to 5.0 |
| Colloidal silicon dioxide | 0.2 to 5.0 |
| Opadry clear YS-1-7006 | 2.0 to 20.0 |
| Simethicone USP | 0.2 to 5.0 |
| Opadry | 10.0 to 75.0 |

Shiny Coating

| | |
|---|---|
| Opadry clear YS-1-7006 | 0.2 to 15.0 |
| Purified water | 0.1 to 5.0 |

* Contains 10% in excess

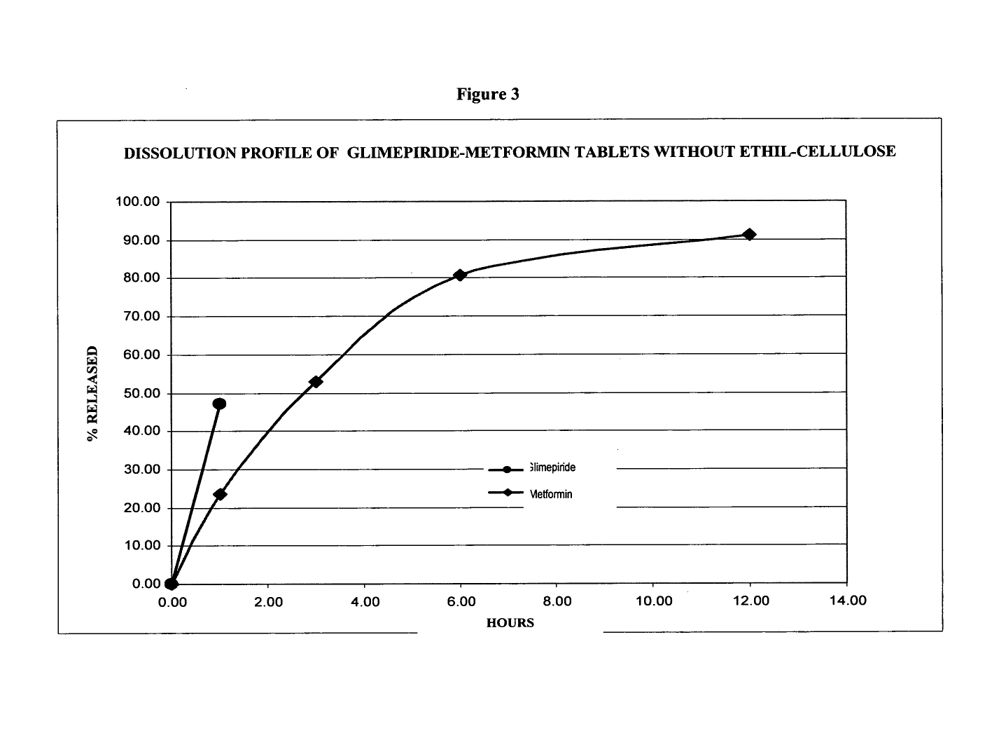

Dissolution tests performed without the insulating coating show that the release profiles of the Metformin HCl located in the core are very similar to those obtained with the insulating coating. However, the release of Glimepiride is affected, releasing only in the range 47-80% (see FIG. 3). These data suggest that due to its ethyl-cellulose insulating coating, the tablet of this invention prevents the diffusion of Glimepiride to the core containing the Metformin HCl, as confirmed in dissolution and stability tests performed, favoring in this way that 100% of the Glimepiride contained is released with the desired rate.

Also as part of this invention, the process for obtaining such composition is described, which, from a technical viewpoint, requires an amount of labor and skill that is amenable to industrial production, which translates to advantages in production costs.

Process for Obtaining the Granulate Conforming the Core of the Tablet with Extended-Release Active:

The process for fabricating the granulate with the formula components for the extended-release core is performed with cutting equipment, preferably one allowing performing all the steps, from mixing the previously-sieved components to granulation, in approximately one hour. The intermediate steps between the mixing of the components and the granulation consist in the addition of the liquid phase (purified water) to the dry mixture at a ratio of 5 to 20 mL/sec. The granulation stage is performed in approximately 7 minutes under standard conditions both in the main mixing equipment as well as in the high-cut mixer. The addition of 20-60% of Hydroxypropyl methyl-cellulose K-100 M is performed in the middle of this 7-minute stage, keeping the thermal balance of the container around 30° C. The granulated material is submitted to the final stage of drying performed in the same cutting equipment during 60 minutes and with a container thermal balance around 50° C. and 70° C. in the lid. The dry stage may be performed under pressure conditions of 50 mbar, with injection of nitrogen gas and application of microwaves, according to recommendations in the cutting equipment used.

Once a granulate with a humidity range between 1.0-3.0 is obtained, in another conventional mixer this mixture is incorporated with 40 to 80% of the residuary Hydroxypropyl methyl-cellulose K-100 M for 3 to 20 minutes at 5 to 20 rpm, with the final addition of magnesium stearate as a lubricant agent for dust for 5 to 10 minutes at 10 to 20 rpm, depending on the recommendations for the mixing equipment.

The final stage for constituting the core of the extended-release tablet is the compression with cylindrical biconcave stamps or caplets, grooved or without groove in high- or low-speed tableting equipments.

Core Coating with an Insulating Layer

This step is performed by dispersion of Opadry clear YS-1-7006 in purified water and the addition of watery dispersion of ethyl-cellulose E-7-7050 clear. The coating protocol is performed according to what is stated in the Integral Coating System GS.

Superficial Coating with Immediate-Release Active Substance

The coating with active drug Glimepiride to be released immediately was performed according to specifications of the Integral Coating System GS by conventional methods of dispersion of the mixture of formulation components listed in table II, starting with Glimepiride in suspension with a final concentration of 20% in excess in order to compensate loss of material during its application. Once the tablets have been coated with the second drug, the Opadry coating was added to make the tablets shine and give them good appearance.

The tablets may be conditioned in different primary packaging, bubble packaging (PVC, PVDC, PET) and flasks (PEAD, Glass, PET) which are subject to stability studies under accelerated conditions following international guidelines established in USP (United States Pharmacopeia 28—National Formulary 23. Rockville, Md.: U.S. Pharmacopeial Convention 2005) and the ICH (International Conference of Harmonization, Guideline for Industry, Stability Testing of New Drug Substances and Products. ICH-Q1A, September 1994).

Other embodiments of the invention will be apparent to those of skill in the art from a consideration of this specification or practice of the invention described herein.

Below, the invention is exemplified by determining a modality of the pharmaceutical composition and a modality of the method for obtaining such tablets, as well as an example of the results of consistent stability studies.

### EXAMPLE 1

The process for obtaining the pharmaceutical composition of the invention consists basically of the following steps:

a) mixing Metformin hydrochloride, microcrystalline cellulose PH 101, Polyvidone K 90 and Colloidal silicon dioxide for 180 seconds in the cutting equipment at 200 rpm and at 600 rpm in high cut;

b) adding the liquid phase (purified water) to the dry mixture at a ratio of 5 to 20 mL/sec;

c) carrying out the granulation for approximately 7 minutes under standard conditions for the equipment both in the main mixer and in the high-cut mixer; the addition of 20-60% of Hydroxypropyl methyl-cellulose K-100 M is performed in the middle of this 7-minute stage, keeping the thermal balance of the container around 30° C.;

d) submitting the granulate material to the final stage of drying that is performed in the same cutting equipment for approximately 60 minutes at a thermal balance of between 50° C. and 70° C. in the lid and with pressure conditions around 50 mbar, with injection of nitrogen gas and application of microwaves, according to recommendations for the cutting equipment used;

e) once the granulate with a humidity range between 1.0-3.0 is obtained, incorporate this mixture in another conventional mixer with 40 to 80% of the residual Hydroxypropyl methyl-cellulose K-100 M for 3 to 20 minutes at 5 to 20 rpm;

f) adding the magnesium stearate as lubricant agent for dust for 5 to 10 minutes at 10 to 20 rpm, depending on the recommendations for the mixing equipment;

g) constituting the core of the extended-release tablet by compression with cylindrical biconcave stamps or caplets, grooved or without groove in high- or low-speed tableting equipments;

h) carrying out the preparation of the insulating coating of the core according to what is stated in the Integral Coating System GS, first dispersing the watery solution containing Opadry clear YS-1-7006 and then dispersing the watery solution Ethyl-cellulose E-7-7050 Clear;

i) performing the coating with immediate-release active ingredient according to what is stated in the Integral Coating System GS through the dispersion of the mixture containing micronized Glimepiride, sodium lauryl sulphate, colloidal silicon dioxide, Opadry clear YS-1-7006 and Simethicone USP with a final concentration of 20% in excess of Glimepiride in order to compensate for loss of material during its application;

j) once the tablets have been coated with the second drug, the Opadry coating was added to make the tablets shine and give them good appearance.

### EXAMPLE 2

Description of the final product once all the steps from example 1 of this document have been performed.

| DETERMINATION | SPECIFICATION (Internal) |
|---|---|
| Description | Grooved oblong tablet color-coated |
| | depending on the concentration of |
| | the product |
| Identification, liquid | Similar to the one obtained with the |
| chromatography Metformin, | reference solution |
| hydrochloride | |
| Assessment | |
| Metformin, hydrochloride | 90.0-110.0% |
| Glimepiride | 1,000, 850 mg/tablet 2, 4 mg/tablet |
| Dose uniformity | |
| Glimepiride | 85-115% |
| Relative standard deviation | No more than 6.0% |
| Related substances | |
| Metformin, hydrochloride | No more than 0.02% |
| for Cyanoguanidine | |
| Related substances | |
| Metformin, hydrochloride | No more than 0.1% |
| for any other impurity | |
| Dissolution | |
| Metformin HCl | |
| 1 hour | 15-35% |
| 3 hours | 40-70% |
| 6 hours | 70-90% |

### EXAMPLE 3

Presentations Formulated for the Final Product

| Proportion of Immediate- | Proportion of extended-release |
|---|---|
| release Glimepiride. | Metformin. |
| 2 | 1,000 |
| 4 | 1,000 |
| 2 | 850 |
| 4 | 850 |
| 1 | 500 |

### EXAMPLE 4

Stability studies performed on the pharmaceutical compositions manufactured according to the present invention were performed in selected temperature and humidity conditions of 40° C. and 75% relative humidity. The test was performed for a period of 6 months, proving that the pharmaceutical composition is stable, just as shown in the results in table II.

TABLE II

| | | TIME | | | | |
|---|---|---|---|---|---|---|
| DETERMINATION | SPECIFICATIONS | INITIAL | 1 MONTH | 2 MONTHS | 3 MONTHS | 6 MONTHS |
| DESCRIPTION | Grooved oblong tablet, with | Correct | Correct | Correct | Correct | Correct |
| | logotype, coated in orange. | | | | | |
| CONTENTS OF | 90.0%-110.0% | 98.16% | 97.81% | 100.34% | 99.40% | 100.34% |
| METFORMIN HCL | | | | | | |
| CONTENTS OF | 90.0%-110.0% | 106.89% | 106.89% | 99.56% | 100.03% | 102.50% |
| GLIMEPIRIDE | | | | | | |
| DISSOLUTION OF | | | | | | |
| METFORMIN HCL, | | | | | | |
| HYDROCHLORIDE | | | | | | |
| 1^{st} hour | 15.0-35.0% | 20.20% | 25.60% | 22.40% | 18.40% | 23.60% |
| 3^{rd} hour | 40.0-70.0% | 51.30% | 59.60% | 54.90% | 54.40% | 56.60% |
| 6^{th} hour | 70.0-90.0% | 79.60% | 89.40% | 83.30% | 86.20% | 85.20% |
| 12^{th} hour | >80.0% | 95.00% | 101.60% | 96.80% | 98.20% | 99.60% |
| DISSOLUTION OF | Q = 60% | 100.80% | 102.66% | 97.41% | 93.33% | 99.53% |
| GLIMEPIRIDE | | | | | | |
| RELATED | No more than 0.02% of | <0.02% | <0.02% | <0.02% | <0.02% | <0.02% |
| SUBSTANCES | Cyanoguanidine | | | | | |
| | No more than 0.1% of other | <0.1% | <0.1% | <0.1% | <0.1% | <0.1% |
| | impurities | | | | | |

Everything that has been exemplified has illustrative purposes. Any person with expertise in the technical field of the invention may know it is possible to include other drugs, and also that other forms, components, additives, proportions, formulation methods and other parameters described herein may be modified or replaced by a variety of options, always in the light of the invention description.

1. A stable pharmaceutical composition in the form of a tablet comprising a core or matrix containing an extended-release biguanide; further comprising an insulating layer or coating comprising a hydrophobic polymer, and further comprising a coating containing an immediate-release sulphonylurea.

2. The pharmaceutical composition of claim 1, wherein said core or matrix is comprises around 250 to around 1,500 mg of a biguanide.

3. The pharmaceutical composition of claim 1, wherein the cover comprises around 0.2 to around 10 mg of a sulphonylurea.

4. The pharmaceutical composition of claim 1, wherein the insulating intermediate layer between the core and the coating comprises around 5 to around 50 mg of ethyl-cellulose.

5. The pharmaceutical composition of claim 1 or 2, wherein said biguanide is selected from the group consisting of: metformin, fenformin and buformin.

6. The pharmaceutical composition of claim 5, wherein said compsition comprises around 250 to around 1,000 mg of metformin hydrochloride.

7. The pharmaceutical composition of claim 1 or 3, wherein said sulphonylurea is optionally selected from the group consisting of: glimepiride, glipizide, glyburide, glibornuride, glisoxepide, gliclazide, acetohexamide, clopropamide, tolazamide and tolbutamide.

8. The pharmaceutical composition of claim 7, wherein said composition comprises about 0.2 to about 10.0 mg of glimepiride.

9. The pharmaceutical composition of claim 1, wherein said core or matrix containing a biguanide further comprises: around 10 to around 100 mg of microcrystalline cellulose; about 30 to about 150 mg of polyvidone K 90; about 150 to about 500 mg of hydroxypropyl methyl-cellulose K-100 M; about 1 to about 50 mg of colloidal silicon dioxide; and about 5 to about 50 mg of magnesium stearate.

10. The pharmaceutical composition of claim 1, wherein said insulating coating further comprises about 0.2 to about 10 mg of opadry clear YS-1-7006.

11. The pharmaceutical composition of claim 1, wherein said coating containing the sulphonylurea further comprises: about 0.2 to about 5 mg of sodium lauryl sulphate; about 0.2 to about 5 mg of colloidal silicon dioxide; about 2 to about 20 mg of opadry; about 2 to about 5 mg of simethicone; about 10 to about 75 mg of opadry color; and optionally a shiny coating containing about 0.2 to about 15 mg of opadry clear and about 0.1 to about 5 mg of purified water.

12. A process for obtaining the pharmaceutical composition of claim 1, comprising the following steps:
a) mixing the Metformin hydrochloride, microcrystalline cellulose PH 101, Polyvidone K 90 and Colloidal silicon dioxide for about 180 seconds in the cut equipment at approximately 200 rpm and at approximately 600 rpm in high cut;
b) adding the liquid phase (purified water) to dry mixture at a ratio of about 5 to 20 mL/sec;
c) carrying out the granulation for approximately 7 minutes under standard conditions for the equipment both in the main mixer and in the high-cut mixer, wherein the addition of approximately 20-60% of Hydroxypropyl methyl-cellulose K-100 M is performed in the middle of this 7-minute stage, keeping a thermal balance of around 30° C.;
d) submitting the granulate material to the final stage of drying that is performed in the same cutting equipment for approximately 60 minutes with a thermal balance between about 50° C. and about 70° C. in the lid, wherein the pressure conditions are around 50 mbar, and wherein injection of nitrogen gas and application of microwaves is maintained, according to recommendations for the cutting equipment used;
e) once the granulate with a humidity range between about 1.0 to about 3.0 is obtained, incorporating the granulate mixture in another conventional mixer with about 40 to about 80% of the residuary Hydroxypropyl methyl-cellulose K-100 M for approximately 3 to 20 minutes at about 5 to about 20 rpm;
f) adding the magnesium stearate as lubricant agent for dust for approximately 5 to 10 minutes at 10 to 20 rpm, depending on the recommendations for the mixing equipment;
g) constituting the core of the extended-release tablet by compression with cylindrical biconcave stamps or caplets, grooved or without groove in high- or low-speed tableting equipment;
h) carrying out insulating the coating of the core according to what is stated in the Integral Coating System GS, by first dispersing the watery solution containing Opadry clear YS-1-7006 and then dispersing the watery solution Ethyl-cellulose E-7-7050 Clear;
i) performing the coating with immediate-release active according to what is stated in the Integral Coating System GS through the dispersion of the mixture containing micronized Glimepiride, sodium lauryl sulphate, colloidal silicon dioxide, Opadry clear YS-1-7600 and Simethicone USP for a final concentration of 20% in excess of Glimepiride in order to compensate for any loss of material during its application;
j) once the tablets have been coated with the second drug, adding the Opadry coating to make the tablets shiny and give them good appearance.

13. The pharmaceutical composition of claim 1, wherein said composition is suitable for administration once or twice per day at dosages of between about 250 to about 1,500 mg of a biguanide and about 0.2 to about 10 mg of a sulphonylurea, for the treatment of diabetes, type 2.
